SEQUENCE LISTING (12 sequences)

SEQ ID NO: 1 - DNA, 1083 bp, synthetic construct
  source 1..1083: mol_type = other DNA
aactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaatacc

SEQ ID NO: 2 - RNA, 19 nt, synthetic construct
  source 1..19: mol_type = other RNA
accaactaaccccctaata

SEQ ID NO: 3 - RNA, 21 nt, synthetic construct
  source 1..21: mol_type = other RNA
cctaaggttaagtcgccctcg

SEQ ID NO: 4 - DNA, 1141 bp, synthetic construct
  source 1..1141: mol_type = other DNA
atgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 5 - DNA, 18 bp, synthetic construct
  source 1..18: mol_type = other DNA
agcattggacagtagcat

SEQ ID NO: 6 - DNA, 18 bp, synthetic construct
  source 1..18: mol_type = other DNA
gagccgaagtttcatcat

SEQ ID NO: 7 - DNA, 20 bp, synthetic construct
  source 1..20: mol_type = other DNA
tgacgtggacatccgcaaag

SEQ ID NO: 8 - DNA, 20 bp, synthetic construct
  source 1..20: mol_type = other DNA
ctggaaggtggacagcgagg

SEQ ID NO: 9 - DNA, 24 bp, synthetic construct
  source 1..24: mol_type = other DNA
tcacaacaatcctaatcctaatac

SEQ ID NO: 10 - DNA, 18 bp, synthetic construct
  source 1..18: mol_type = other DNA
catgcggagatgttggat

SEQ ID NO: 11 - DNA, 20 bp, synthetic construct
  source 1..20: mol_type = other DNA
tttcgcccactaagccaatc

SEQ ID NO: 12 - DNA, 21 bp, synthetic construct
  source 1..21: mol_type = other DNA
gcccatttgagtattttgttt

(19) United States
(12) Patent Application Publication
(10) Pub. No.: US 2025/0283087 A1
(43) Pub. Date: Sep. 11, 2025

(54) ISOLATED CIRCULAR RNA AND USE THEREOF IN PREVENTING AND TREATING LUNG CANCER

(71) Applicant: Guangzhou Institutes of Biomedicine and Health, Chinese Academy of Sciences, Guangzhou (CN)

(72) Inventors: Xingguo LIU, Guangzhou (CN); Zifeng RUAN, Guangzhou (CN); Liang YANG, Guangzhou (CN)

(73) Assignee: Guangzhou Institutes of Biomedicine and Health, Chinese Academy of Sciences, Guangzhou (CN)

(21) Appl. No.: 19/220,119

(22) Filed: May 28, 2025

Related U.S. Application Data
(63) Continuation of application No. PCT/CN2022/141065, filed on Dec. 22, 2022.

(51) Int. Cl. C12N 15/113; C12Q 1/6886

(52) U.S. Cl. CPC C12N 15/1135; C12Q 1/6886; C12N 2310/113; C12N 2310/531; C12Q 2600/178

(57) ABSTRACT

Provided are an isolated circular nucleic acid molecule and a pharmaceutical composition. The nucleotide sequence of the isolated circular nucleic acid molecule is set forth in SEQ ID NO: 1. The pharmaceutical composition comprises: a reagent for inhibiting the expression or activity of a circular nucleic acid molecule, the circular nucleic acid molecule having a nucleotide sequence as set forth in SEQ ID NO: 1.

### CROSS-REFERENCE TO RELATED APPLICATIONS

This application is a Continuation of PCT Patent Application No. PCT/CN2022/141065 having International filing date of Dec. 22, 2022, the contents of which are all incorporated by reference as if fully set forth herein in their entirety.

### SEQUENCE LISTING STATEMENT

The XML file, entitled 103958SequenceListing.xml, created on May 20, 2025, comprising 13,436 bytes, submitted concurrently with the filing of this application is incorporated herein by reference.

### FIELD AND BACKGROUND OF THE INVENTION

The present application relates to the field of bioengineering and, in particularly, relates to an isolated circular RNA and use thereof in preventing and treating lung cancer, more particularly to an isolated circular nucleic acid molecule, an isolated protein or polypeptide, a construct, a recombinant cell, use of a reagent in the manufacture of a medicament, a pharmaceutical composition and use thereof.

Cancer has become the leading cause of death, with a mortality rate of 130.7 deaths per 100,000 people. The top five cancers with the highest mortality rates, in descending order, are lung cancer, gastric cancer, liver cancer, colorectal cancer, and pancreatic cancer. These five cancers account for approximately 70% of all cancer deaths. Among men, the mortality rates for lung cancer, gastric cancer, liver cancer, and colorectal cancer make up 70% of all cancer deaths in males. Among women, gastric cancer, lung cancer, liver cancer, colorectal cancer, and pancreatic cancer make up 60% of all cancer deaths in females.

Lung cancer is a malignant tumor originating from the bronchial mucosa or glands of the lungs. It has the fastest-growing incidence and mortality rates, and is one of the greatest threats to public health and life. Early-stage lung cancer often presents with no obvious symptoms, which can lead to it being overlooked. By the time clinical symptoms appear, the disease is often already in its advanced stages, making surgery unfeasible. This highlights the importance of early detection and treatment of lung cancer. However, there is currently a lack of targeted and specific treatments for early-stage lung cancer. Therefore, finding a specific drug or method for treating early-stage lung cancer is crucial for its effective treatment.

### SUMMARY OF THE INVENTION

The present disclosure aims to solve at least one of the technical problems in the related art to a certain extent. To this end, one object of the present disclosure is to provide an isolated CircCYB and use thereof.

The present disclosure was accomplished based on the following findings of the inventors:

The human CYTB gene is located on the mitochondrial genome (position: 14737 . . . 15877) and encodes the respiratory chain protein CYTB. The inventors, through experimentation, first discovered that in multiple human cell types (such as embryonic stem cells, induced pluripotent stem cells, and cancer cells), the CYTB gene can be transcribed to form a circular RNA of 1083 bp in length, which is a head-to-tail linked molecule known as circular CYTB (also referred to as CircCYB). The inventors also made an exciting discovery through experimentation: knocking down the expression level of CircCYB in cancer cells (particularly in lung cancer cells) promotes apoptosis and cell death of lung cancer cells without affecting the level of linear CYTB mRNA, and with no apoptotic effect on normal cells. Therefore, by regulating the expression level of CircCYB in cancer cells, it is possible to specifically control apoptosis in cancer cells. The nucleotide sequence of the human CYTB gene is set forth in SEQ ID NO: 4, with the CircCYB sequence underlined.

(SEQ ID NO: 4)
ATGACCCCAATACGCAAAACTAACCCCCTAATAAAATTAATTAACCACT
CATTCATCGACCTCCCCACCCCATCCAACATCTCCGCATGATGAAACTT
CGGCTCACTCCTTGGCGCCTGCCTGATCCTCCAAATCACCACAGGACTA
TTCCTAGCCATGCACTACTCACCAGACGCCTCAACCGCCTTTTCATCAA
TCGCCCACATCACTCGAGACGTAAATTATGGCTGAATCATCCGCTACCT
TCACGCCAATGGCGCCTCAATATTCTTTATCTGCCTCTTCCTACACATC
GGGCGAGGCCTATATTACGGATCATTTCTCTACTCAGAAACCTGAAACA
TCGGCATTATCCTCCTGCTTGCAACTATAGCAACAGCCTTCATAGGCTA
TGTCCTCCCGTGAGGCCAAATATCATTCTGAGGGGCCACAGTAATTACA
AACTTACTATCCGCCATCCCATACATTGGGACAGACCTAGTTCAATGAA
TCTGAGGAGGCTACTCAGTAGACAGTCCCACCCTCACACGATTCTTTAC
CTTTCACTTCATCTTGCCCTTCATTATTGCAGCCCTAGCAACACTCCAC
CTCCTATTCTTGCACGAAACGGGATCAAACAACCCCCTAGGAATCACCT
TTCTCACCAGACCTCCTAGGCGACCCAGACAATTATACCCTAGCCAACC
CCCCATTCCGATAAAATCACCTTCCACCCTTACTACACAATCAAAGACG
CCCTCGGCTTACTTCTCTTCCTTCTCTCCTTAATGACATTAACACTACT
TAAACACCCCTCCCCACATCAAGCCCGAATGATATTTCCTATTCGCCTA
CACAATTCTCCGATCCGTCCCTAACAAACTAGGAGGCGTCCTTGCCCTA
TTACTATCCATCCTCATCCTAGCAATAATCCCCATCCTCCATATATCCA
AACAACAAAGCATAATATTTCGCCCACTAAGCCAATCACTTTATTGACT
CCTAGCCGCAGACCTCCTCATTCTAACCTGAATCGGAGGACAACCAGTA
AGCTACCCTTTTACCATCATTGGACAAGTAGCATCCGTACTATACTTCA
CAACAATCCTAATCCTAATACCAACTATCTCCCTAATTGAAAACAAAAT
ACTCAAATGGGCCT.

In one aspect of the present disclosure, provided is an isolated circular nucleic acid molecule. According to an embodiment of the present disclosure, the isolated circular nucleic acid molecule has a nucleotide sequence as set forth in SEQ ID NO: 1. The inventors have experimentally found that the isolated circular nucleic acid molecule (i.e., CircCYB) plays an important role in regulating cancer progression.

In another aspect of the present disclosure, provided is an isolated protein or polypeptide. According to an embodiment of the present disclosure, the isolated protein or polypeptide is encoded by the aforementioned isolated circular nucleic acid molecule. Thus, the aforementioned isolated circular nucleic acid molecule can be used to encode the isolated protein or polypeptide.

In yet another aspect of the present disclosure, provided is a construct. According to an embodiment of the present disclosure, the construct carries the aforementioned isolated circular nucleic acid molecule. The construct can be effectively used to express the aforementioned isolated protein or polypeptide, especially in prokaryotic or lower eukaryotic expression systems for efficient expression of the aforementioned isolated protein or polypeptide.

In yet another aspect of the present disclosure, provided is a recombinant cell. According to an embodiment of the present disclosure, the recombinant cell carries the aforementioned isolated circular nucleic acid molecule or the aforementioned construct, or expresses the aforementioned isolated protein or polypeptide. The recombinant cell according to an embodiment of the present disclosure can be used for the in vitro expression and large-scale production of the aforementioned protein or polypeptide.

In yet another aspect of the present disclosure, provided is use of a reagent in the manufacture of a medicament for preventing and/or treating a cancer or tumor. The reagent is used for inhibiting the expression or activity of a circular nucleic acid molecule having a nucleotide sequence as set forth in SEQ ID NO: 1. The inventors have experimentally found that the use of the aforementioned reagent can promote apoptosis and cell death of cancer cells and can effectively treat or prevent cancer or tumors.

In yet another aspect of the present disclosure, provided is use of a reagent in the manufacture of a kit for detecting a cancer or tumor. The reagent is used for detecting a circular nucleic acid molecule having a nucleotide sequence as set forth in SEQ ID NO: 1. The inventors have experimentally found that the aforementioned circular nucleic acid molecule can be highly expressed in cancer cells. Thus, the aforementioned reagent can be used to detect cancer or tumor by detecting the expression level of the circular nucleic acid molecule in cancer cells.

In yet another aspect of the present disclosure, provided is a pharmaceutical composition. According to an embodiment of the present disclosure, the pharmaceutical composition includes a reagent that inhibits the expression or activity of a circular nucleic acid molecule having a nucleotide sequence as set forth in SEQ ID NO: 1. The inventors have found through extensive experiments that the aforementioned pharmaceutical composition can effectively promote apoptosis and cell death of lung cancer cells; alternatively, the aforementioned pharmaceutical composition is used for promoting apoptosis in cancer cells when culturing cells in vitro.

In yet another aspect of the present disclosure, provided is use of a pharmaceutical composition in the manufacture of a medicament for preventing and/or treating a cancer or tumor. As mentioned above, the CircCYB gene plays an important role in regulating cancer progression. Thus, the aforementioned pharmaceutical composition including a reagent that knocks down the CircCYB gene in cancer cells can effectively treat or prevent cancer or tumors.

In yet another aspect of the present disclosure, provided is a method for promoting apoptosis in a cancer cell. According to an embodiment of the present disclosure, the method includes contacting a cancer cell expressing a circular nucleic acid molecule with a reagent for inhibiting the expression or activity of the circular nucleic acid molecule. The circular nucleic acid molecule has a nucleotide sequence as set forth in SEQ ID NO: 1. The inventors have experimentally found that contacting cancer cells with the aforementioned reagent promotes apoptosis in cancer cells when culturing cancer cells in vitro.

In yet another aspect of the present disclosure, provided is a method for screening a medicament for treating or preventing cancer, the method including: contacting a medicament to be screened with a cancer cell, and determining a target medicament based on a change in a circular nucleic acid molecule having a nucleotide sequence as set forth in SEQ ID NO: 1 in the cancer cell before and after said contacting. As mentioned above, the aforementioned circular nucleic acid molecule can be highly expressed in cancer cells. Thus, the method can be used for screening medicaments by detecting the expression level of the circular nucleic acid molecule in cancer cells according to the changes in the circular nucleic acid molecule.

In yet another aspect of the present disclosure, provided is a method for preventing and/or treating a cancer or tumor. According to an embodiment of the present disclosure, the method includes administering to a subject a pharmaceutically acceptable amount of the aforementioned pharmaceutical composition. The method of the present disclosure can effectively prevent and/or treat a cancer or tumor.

### BRIEF DESCRIPTION OF THE DRAWINGS

FIG. 1 is a graph showing the presence of CircCYB in human H1 cells, GZF2-ipsc cells, A549 and H1299 cells verified in Example 1 of the present disclosure;

FIG. 2 is a graph showing the presence of CircCYB using the RNase R experiment verified in Example 2 of the present disclosure;

FIG. 3 is a graph showing the relative RNA expression level in human lung adenocarcinoma cancer and paracancerous tissues, as well as lung cancer cells and normal lung epithelial cells detected in Example 3 of the present disclosure;

FIG. 4 is a graph showing the relative RNA expression level in the constructed CircCYB-knockdown human lung adenocarcinoma A549 and H1299 cells detected in Example 4 of the present disclosure;

FIG. 5 is a graph showing the apoptosis in CircCYB-knockdown human lung adenocarcinoma A549 and H1299 cells detected in Example 5 of the present disclosure;

FIG. 6 is a graph showing the apoptosis in CircCYB-knockdown human normal lung epithelial BESA-2B cells detected in Example 6 of the present disclosure; and

FIG. 7 is a graph showing the number of CircCYB-knockdown human lung adenocarcinomas A549 and H1299 cells detected in Example 7 of the present disclosure.

### DESCRIPTION OF SPECIFIC EMBODIMENTS OF THE INVENTION

Hereinafter, embodiments of the present disclosure will be described in detail. The embodiments described below are illustrative only and are not to be construed as limiting the present disclosure.

It should be noted that the terms “first” and “second” are used for descriptive purposes only and are not to be understood as indicating or implying relative importance or implicitly indicating the number of technical features indicated. Thus, a feature defined as “first” or “second” may explicitly or implicitly include one or more of the features. Further, in the description of the present disclosure, unless otherwise specified, the meaning of “more” is two or more.

As used herein, the terms “including” or “comprising” are used in an open-ended fashion, i.e. to include what is specified in the present disclosure, but not to exclude other aspects.

As used herein, the terms “optionally”, “optional” or “option” generally mean that the subsequently described event or circumstance may but need not occur and that the description includes instances where the event or circumstance occurs and instances in which it does not.

As used herein, the term “pharmaceutical composition” may refer to the treatment of a disease or may be used in an in vitro culture experiment of cells. The term “pharmaceutical composition” as used in the treatment of diseases refers generally to unit dosage forms and may be prepared by any of the methods well known in the pharmaceutical art. All methods include the step of bringing the active ingredient into association with the excipient which constitutes one or more accessory ingredients. In general, the composition is prepared by uniformly and fully bringing into association the active compound with a liquid excipient, finely divided solid excipient, or both.

As used herein, the term “pharmaceutically acceptable” means that the substance or composition must be compatible chemically and/or toxicologically, with the other ingredients included in a formulation, and/or the mammal being treated therewith. Preferably, “pharmaceutically acceptable” as used herein means approved by a federal regulatory agency or a national government or listed in the U.S. Pharmacopeia or other generally recognized pharmacopeia for use in animals, and particularly in humans.

As used herein, the term “pharmaceutically acceptable excipient” or “pharmaceutically acceptable carrier” can include any solvent, solid excipient, diluent, or other liquid excipient, etc. suitable for the particular desired dosage form. Except insofar as any conventional excipient is incompatible with the compounds of the present disclosure, such as by producing any undesirable biological effect or interacting in a deleterious manner with any other component(s) of the pharmaceutically acceptable composition, its use is contemplated to be within the scope of the present disclosure.

For further pharmaceutically acceptable excipients mentioned herein and processes thereof, reference is made to the extensive literature on this subject, in particular to Handbook of Pharmaceutical Excipients, 3rd edition, edited by Arthur H. Kibbe, American Pharmaceutical Association, Washington, USA and Pharmaceutical Press, London; and Lexikon der Hilfsstoffe für Pharmazie, edited by Kosmetik and angrenzende Gebiete, H. P. Fiedler, 4th edition, edited by Cantor, Aulendorf and earlier editions.

As used herein, the term “cancer” or “tumor” can be any unregulated cell growth. Illustratively, it may be non-small cell lung cancer, papillary thyroid carcinoma, glioblastoma multiforme, colon cancer, rectal cancer, lung cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, neuroblastoma, pancreatic cancer, melanoma, head and neck squamous cell carcinoma, cervical cancer, skin cancer, glioma, esophageal cancer, oral squamous cell carcinoma or gastric cancer, and the like.

As used herein, the term “treating” or “treatment” refers to obtaining a desired pharmacological and/or physiological effect. Said effect may be prophylactic in terms of complete or partial prevention of the disease or its symptoms, and/or may be therapeutic in terms of partial or complete cure of the disease and/or adverse effects caused by the disease. The “treating” or “treatment” as used herein encompasses the treatment of diseases in mammals, especially human, including: (a) prevention of diseases in individuals who are prone to diseases but have not yet been diagnosed; (b) inhibition of diseases, such as blocking the progression of the disease; or (c) alleviation of diseases, such as reducing symptoms associated with the disease. The “treating” or “treatment” as used herein encompasses any medication that administers a medicament or compound to an individual to treat, cure, alleviate, ameliorate, reduce or inhibit the individual's disease, including but not limited to administering the medicament including the compound as described herein to the individual in need thereof.

The present disclosure provides an isolated circular nucleic acid molecule, an isolated protein or polypeptide, a construct, a recombinant cell, a reagent for use in the manufacture of a medicament, a pharmaceutical composition and use thereof.

### Isolated Circular Nucleic Acid Molecule, Isolated Protein or Polypeptide, Construct and Recombinant Cell

In one aspect of the present disclosure, an isolated circular nucleic acid molecule is provided. According to an embodiment of the present disclosure, the isolated circular nucleic acid molecule has a nucleotide sequence as set forth in SEQ ID NO: 1. The inventors have experimentally found that the isolated circular nucleic acid molecule (i.e., CircCYB) plays an important role in regulating cancer progression.

It should be noted that the structure of the isolated circular nucleic acid molecule is a head-to-tail linked circular structure formed by transcription followed by splicing of the nucleotide sequence as set forth in SEQ ID NO: 1.

(SEQ ID NO: 1)
AACTAACCCCCTAATAAAATTAATTAACCACTCATTCATCGACCTCCCC
ACCCCATCCAACATCTCCGCATGATGAAACTTCGGCTCACTCCTTGGCG
CCTGCCTGATCCTCCAAATCACCACAGGACTATTCCTAGCCATGCACTA
CTCACCAGACGCCTCAACCGCCTTTTCATCAATCGCCCACATCACTCGA
GACGTAAATTATGGCTGAATCATCCGCTACCTTCACGCCAATGGCGCCT
CAATATTCTTTATCTGCCTCTTCCTACACATCGGGCGAGGCCTATATTA
CGGATCATTTCTCTACTCAGAAACCTGAAACATCGGCATTATCCTCCTG
CTTGCAACTATAGCAACAGCCTTCATAGGCTATGTCCTCCCGTGAGGCC
AAATATCATTCTGAGGGGCCACAGTAATTACAAACTTACTATCCGCCAT
CCCATACATTGGGACAGACCTAGTTCAATGAATCTGAGGAGGCTACTCA
GTAGACAGTCCCACCCTCACACGATTCTTTACCTTTCACTTCATCTTGC
CCTTCATTATTGCAGCCCTAGCAACACTCCACCTCCTATTCTTGCACGA
AACGGGATCAAACAACCCCCTAGGAATCACCTCCCATTCCGATAAAATC
ACCTTCCACCCTTACTACACAATCAAAGACGCCCTCGGCTTACTTCTCT
TCCTTCTCTCCTTAATGACATTAACACTATTCTCACCAGACCTCCTAGG
CGACCCAGACAATTATACCCTAGCCAACCCCTTAAACACCCCTCCCCAC
ATCAAGCCCGAATGATATTTCCTATTCGCCTACACAATTCTCCGATCCG
TCCCTAACAAACTAGGAGGCGTCCTTGCCCTATTACTATCCATCCTCAT
CCTAGCAATAATCCCCATCCTCCATATATCCAAACAACAAAGCATAATA
TTTCGCCCACTAAGCCAATCACTTTATTGACTCCTAGCCGCAGACCTCC
TCATTCTAACCTGAATCGGAGGACAACCAGTAAGCTACCCTTTTACCAT
CATTGGACAAGTAGCATCCGTACTATACTTCACAACAATCCTAATCCTA
ATACC.

It should be noted that for the nucleic acid molecules referred to herein, one of the skills in the art would understand that either or both of the complementary duplexes are actually included. For convenience, in this specification and claims, while in most cases only one strand is shown, in fact, another strand complementary thereto is also disclosed. In addition, the nucleic acid sequences of the present application include DNA forms or RNA forms, and disclosing one of them means that the other is also disclosed.

In another aspect of the present disclosure, the present disclosure provides an isolated protein or polypeptide. According to an embodiment of the present disclosure, the isolated protein or polypeptide is encoded by the aforementioned isolated circular nucleic acid molecule. Thus, the aforementioned isolated circular nucleic acid molecule can be used to encode the isolated protein or polypeptide.

In yet another aspect of the present disclosure, provided is a construct. According to an embodiment of the present disclosure, the construct carries the aforementioned isolated circular nucleic acid molecule. The construct can be effectively used to express the aforementioned isolated protein or polypeptide, especially in prokaryotic or lower eukaryotic expression systems for efficient expression of the aforementioned isolated protein or polypeptide.

According to an embodiment of the present disclosure, the vector of the construct is a non-pathogenic viral vector.

According to an embodiment of the present disclosure, the vector of the construct is an adenoviral vector, a lentiviral vector, or a retroviral vector.

In yet another aspect of the present disclosure, provided is a recombinant cell. According to an embodiment of the present disclosure, the recombinant cell carries the aforementioned isolated circular nucleic acid molecule or the aforementioned construct, or expresses the aforementioned isolated protein or polypeptide. The recombinant cell according to an embodiment of the present disclosure can be used for the in vitro expression and large-scale production of the aforementioned protein or polypeptide.

### Uses

In yet another aspect of the present disclosure, provided is use of a reagent in the manufacture of a medicament for preventing and/or treating a cancer or tumor. The reagent is used for inhibiting the expression or activity of a circular nucleic acid molecule having a nucleotide sequence as set forth in SEQ ID NO: 1. The inventors have experimentally found that the use of the aforementioned reagent can promote apoptosis and cell death of cancer cells and can effectively treat or prevent cancer or tumors

According to an embodiment of the present disclosure, the reagent includes at least one of shRNA, siRNA, and CRISPR/Cas9 for knocking down the circular nucleic acid molecule. As mentioned above, the CircCYB gene plays an important role in regulating cancer progression. Thus, using the aforementioned reagent can knock down the CircCYB gene in cancer cells, thereby effectively treating or preventing cancer or tumors.

As used herein, “siRNA”, “small interfering RNA”, “short interfering RNA” and “silencing RNA” all refer to a double-stranded RNA, which is mainly involved in RNA interference (RNAi) and regulates gene expression in a specific manner. Note that, since the CircCYB gene of the present disclosure is disclosed herein, siRNA can be obtained by conventional methods in the art. By cloning the obtained siRNA into an expression vector and introducing the expression vector into a host cell, the CircCYB gene can be knocked down.

As used herein, the terms “shRNA” and “short hairpin RNA” both refer to a hairpin structure including two short inverted repeats and a stem-loop sequence separating them. It is controlled by a pol III promoter, followed by the addition of 5-6 T as a transcriptional terminator for RNA polymerase III. Note that, since the CircCYB gene of the present disclosure is disclosed herein, shRNA can be obtained by conventional methods in the art. By cloning the obtained shRNA into an expression vector and introducing the expression vector into a host cell, the CircCYB gene can be knocked down.

As used herein, the term “CRISPR/Cas9 vector” includes a gene encoding Cas9 protein and a gene encoding gRNA (or sgRNA). The gene encoding Cas9 protein and the gene encoding gRNA (or sgRNA) may be located on an identical vector or separated vectors. By introducing the CRISPR/Cas9 vector into a host cell, the CircCYB gene can be knocked down. Note that, since the CircCYB gene of the present disclosure is disclosed herein, gRNA (or sgRNA) can be obtained by conventional methods in the art.

Illustratively, the host cell can be a cancer cell or cancer cell line. For example, the cancer cell line is A549 and H1299 cells.

According to an embodiment of the present disclosure, the reagent includes a nucleotide sequence as set forth in SEQ ID NO: 2.

(SEQ ID NO: 2)
ACCAACTAACCCCCTAATA.

According to an embodiment of the present disclosure, the cancer includes lung cancer, non-small cell lung cancer, papillary thyroid cancer, glioblastoma multiforme, colon cancer, rectal cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, neuroblastoma, pancreatic cancer, melanoma, head and neck squamous cell carcinoma, cervical cancer, skin cancer, glioma, esophageal cancer, oral squamous cell carcinoma or gastric cancer.

In yet another aspect of the present disclosure, provided is use of a reagent in the manufacture of a kit for detecting a cancer or tumor. The reagent is used for detecting a circular nucleic acid molecule having a nucleotide sequence as set forth in SEQ ID NO: 1. The inventors have experimentally found that the aforementioned circular nucleic acid molecule can be highly expressed in cancer cells. Thus, the aforementioned reagent can be used to detect cancer or tumor by detecting the expression level of the circular nucleic acid molecule in cancer cells.

According to an embodiment of the present disclosure, the reagent includes a probe, primer, or antibody binding to the circular nucleic acid molecule.

According to an embodiment of the present disclosure, the cancer includes lung cancer, non-small cell lung cancer, papillary thyroid cancer, glioblastoma multiforme, colon cancer, rectal cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, neuroblastoma, pancreatic cancer, melanoma, head and neck squamous cell carcinoma, cervical cancer, skin cancer, glioma, esophageal cancer, oral squamous cell carcinoma or gastric cancer.

### Pharmaceutical Composition and Use Thereof

In yet another aspect of the present disclosure, provided is a pharmaceutical composition. According to an embodiment of the present disclosure, the pharmaceutical composition includes a reagent that inhibits the expression or activity of a circular nucleic acid molecule having a nucleotide sequence as set forth in SEQ ID NO: 1. The inventors have found through extensive experiments that the aforementioned pharmaceutical composition can effectively promote apoptosis and cell death of lung cancer cells; alternatively, the aforementioned pharmaceutical composition is used for promoting apoptosis in cancer cells when culturing cells in vitro.

According to an embodiment of the present disclosure, the pharmaceutical composition further includes a pharmaceutically acceptable excipient.

According to an embodiment of the present disclosure, the reagent includes at least one of shRNA, siRNA, and CRISPR/Cas9 for knocking down the circular nucleic acid molecule.

According to an embodiment of the present disclosure, the reagent includes a nucleotide sequence as set forth in SEQ ID NO: 2.

In yet another aspect of the present disclosure, provided is use of a pharmaceutical composition in the manufacture of a medicament for preventing and/or treating a cancer or tumor. As mentioned above, the CircCYB gene plays an important role in regulating cancer progression. Thus, the aforementioned pharmaceutical composition including a reagent that knocks down the CircCYB gene in cancer cells can effectively treat or prevent cancer or tumors.

According to an embodiment of the present disclosure, the cancer includes lung cancer, non-small cell lung cancer, papillary thyroid cancer, glioblastoma multiforme, colon cancer, rectal cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, neuroblastoma, pancreatic cancer, melanoma, head and neck squamous cell carcinoma, cervical cancer, skin cancer, glioma, esophageal cancer, oral squamous cell carcinoma or gastric cancer.

### Methods

In yet another aspect of the present disclosure, provided is a method for promoting apoptosis in a cancer cell. According to an embodiment of the present disclosure, the method includes contacting a cancer cell expressing a circular nucleic acid molecule with a reagent for inhibiting the expression or activity of the circular nucleic acid molecule. The circular nucleic acid molecule has a nucleotide sequence as set forth in SEQ ID NO: 1. The inventors have experimentally found that contacting cancer cells with the aforementioned reagent promotes apoptosis in cancer cells when culturing cancer cells in vitro.

According to an embodiment of the present disclosure, the reagent includes at least one of shRNA, siRNA, and CRISPR/Cas9 for knocking down the circular nucleic acid molecule.

According to an embodiment of the present disclosure, the reagent includes a nucleotide sequence as set forth in SEQ ID NO: 2.

It should be noted that the cancer cell in the present disclosure includes cancer cells in the body and also includes cancer cell lines, and the specific type is not limited.

Illustratively, the cancer cell line is A549 and H1299 cells.

In yet another aspect of the present disclosure, provided is a method for preventing and/or treating a cancer or tumor. According to an embodiment of the present disclosure, the method includes administering to a subject a pharmaceutically acceptable amount of the aforementioned pharmaceutical composition. The method of the present disclosure can effectively prevent and/or treat a cancer or tumor.

The effective amount of the pharmaceutical composition of the present disclosure may vary depending on, among other things, the mode of administration and the severity of the disease to be treated. Selection of a preferred effective amount can be determined by one of ordinary skill in the art based on a variety of factors (e.g., through clinical trials). Such factors include, but are not limited to: pharmacokinetic parameters of the active ingredient such as bioavailability, metabolism, half-life, and the like; the severity of the disease to be treated in the patient, the weight of the patient, the immune status of the patient, the route of administration, etc. For example, several divided doses may be administered daily or the dose may be proportionally reduced as indicated by the exigencies of the therapeutic situation.

The pharmaceutical composition of the present disclosure may be incorporated into drugs suitable for parenteral administration (e.g., intravenous, subcutaneous, intraperitoneal, intramuscular). These drugs can be prepared in various forms, such as liquid, semisolid, and solid dosage forms, including but are not limited to, liquid solutions (e.g., injection and infusion solutions) or lyophilized powders. Typical drugs are in the form of injection solutions or infusion solutions. The aforementioned pharmaceutical composition may be administered by intravenous infusion or injection, or intramuscular or subcutaneous injection.

According to an embodiment of the present disclosure, the cancer includes lung cancer, non-small cell lung cancer, papillary thyroid cancer, glioblastoma multiforme, colon cancer, rectal cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, neuroblastoma, pancreatic cancer, melanoma, head and neck squamous cell carcinoma, cervical cancer, skin cancer, glioma, esophageal cancer, oral squamous cell carcinoma or gastric cancer.

In yet another aspect of the present disclosure, provided is a method for screening a medicament for treating or preventing cancer, the method including: contacting a medicament to be screened with a cancer cell, and determining a target medicament based on a change in a circular nucleic acid molecule having a nucleotide sequence as set forth in SEQ ID NO: 1 in the cancer cell before and after said contacting. As mentioned above, the aforementioned circular nucleic acid molecule can be highly expressed in cancer cells. Thus, the method can be used for screening medicaments by detecting the expression level of the circular nucleic acid molecule in cancer cells according to the changes in the circular nucleic acid molecule.

According to an embodiment of the present disclosure, a decrease in the expression or activity of the circular nucleic acid molecule in the cancer cell after said contacting is an indicator that the medicament to be screened is the target medicament.

The solutions of the present disclosure will be explained with reference to the following embodiments. It will be understood by those skilled in the art that the following examples are merely illustrative of the present disclosure and are not to be construed as limiting the scope of the present disclosure. Where specific techniques or conditions are not specified in the examples, they are performed according to techniques or conditions described in the literature in the art or according to the product description. The reagents or instruments used are conventional products that can be obtained commercially when not indicating the manufacturer.

### Example 1: PCR Experiments and Sanger Sequencing to Verify the Presence of CircCYB

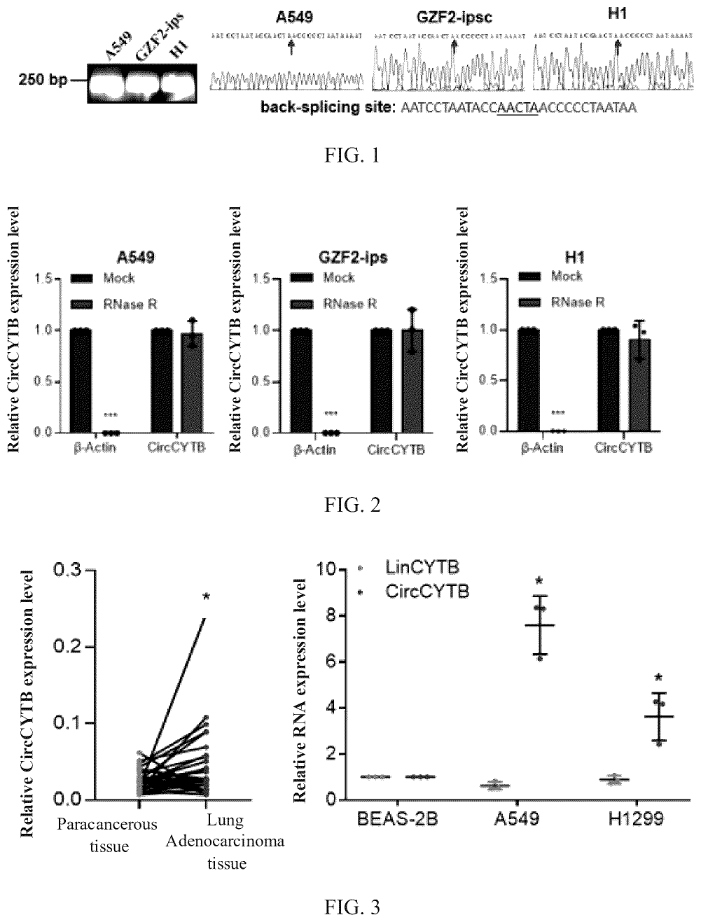

In this example, human H1 cells (human embryonic stem cells), GZF2-ipsc cells (human induced pluripotent stem cells), A549 and H1299 cells (human lung adenocarcinoma cells, also called human lung adenocarcinoma A549 and H1299 cells) were used for verification. RNA was extracted from the above three cell lines and subjected to reverse transcription using the Promega Reverse Transcription System kit to finally obtain cDNA. Reverse splicing PCR primers (forward primer: AGCATTGGACAGTAGCAT (SEQ ID NO: 5); reverse primer: GAGCCGAAGTTTCATCAT (SEQ ID NO: 6)) were designed at the CircCYB interface sequence. A PCR experiment was carried out (according to the KOD DNA polymerase kit, with PCR conditions of 94° C., 2 min→94° C., 15 sec→58° C., 30 sec→68° C., 20 s→68° C., 5 min, for 35 cycles). The resulting PCR product was subjected to agarose gel electrophoresis, and a band of approximately 250 bp was cut and subjected to Sanger sequencing, which confirmed the presence of the CircCYB reverse splicing sequence. The results are shown in FIG. 1. The results showed that CircCYB was present in all three cell types.

### Example 2: RNase R Experiment to Verify the Presence of CircCYB

RNase R is an RNA enzyme that can specifically degrade linear RNA. RNase R can digest most linear RNA under 37° C. conditions for 30 min, while circular RNA can resist digestion by RNase R. Therefore, human H1 cells (human embryonic stem cells), GZF2-ipsc cells (human induced pluripotent stem cells), A549 and H1299 cells (human lung adenocarcinoma cells) in Example 1 were used to verify the presence of CircCYB in the cells. The specific steps are as follows:

RNA was extracted from the aforementioned cell line (the extraction method was the same as in Example 1). Two 2 μg RNA samples were taken. One RNA sample was treated with RNase R (experimental group), while the other RNA sample was treated with an equal volume of nuclease-free water as a control (control group). After treatment at 37° C. for 30 minutes, reverse transcription was performed on both RNA samples using random primers (the reverse transcription method was the same as in Example 1) to obtain cDNA. β-actin was used as a representative of linear RNA, and quantitative real-time PCR primers for β-actin and CircCYB were designed (β-actin-F: TGACGTGGACATCCGCAAAG (SEQ ID NO: 7), β-actin-R: CTGGAAGGTGGACAGCGAGG (SEQ ID NO: 8 8); CircCYB-F: TCACAACAATCCTAATCCTAATAC (SEQ ID NO: 9), CircCYB-R: CATGCGGAGATGTTGGAT (SEQ ID NO: 10)). Finally, quantitative real-time PCR experiments were performed to detect the presence or absence of RNase R and its digestion of β-actin and CircCYB. The experimental system was 20 μL in total volume, and the kit used was GoTaq® qPCR and RT-qPCR Systems (qPCR). The results are shown in FIG. 2. The results showed that CircCYB was resistant to digestion by RNase R and further confirmed the presence of CircCYB.

### Example 3: Expression of CircCYB in Paracancerous Tissues and Human Lung Adenocarcinoma Cells

1. Human lung adenocarcinoma tissues and their paracancerous tissues (paracancerous tissue refers to the tissue located 2 cm away from the lesion, which has the same cell type as the cancer tissue but has not undergone carcinogenesis) collected from the hospital were rapidly frozen in liquid nitrogen and ground in a mortar. The ground tissue powder was transferred to a 1.5 mL centrifuge tube, and then 1 mL of Trizol solution added was added. The mixture was repeatedly pipetted until the powder was fully digested, and then 400 μL of chloroform solution was added and mixed well. After standing at room temperature for 5 minutes, the mixture was centrifuged at 12000×g for 15 minutes at 4° C. After centrifugation, the 400 μL colorless supernatant was carefully transferred to a new 1.5 mL centrifuge tube. An equal volume of isopropanol was added, and the mixture was gently inverted several times to mix. After standing on ice for 10 minutes, the mixture was centrifuged at 12000×g for 10 minutes at 4° C., and the supernatant was discarded. To the pellet, 1 mL of 70% ethanol solution prepared with nuclease-free water was added for washing by vortexing. The mixture was then centrifuged at 12000×g for 5 minutes at 4° C. After removing the supernatant, 50 μL of nuclease-free water was added to dissolve the pellet. 2 μg of RNA was taken for reverse transcription, and the content of CircCYB in human lung adenocarcinoma tissues and their paracancerous tissues was measured (the specific method for CircCYB qPCR detection was the same as in Example 2). The results are shown in FIG. 3. The results indicated that CircCYB expression was higher in human lung adenocarcinoma tissues compared with the paracancerous tissues.
2. The content of CircCYB in normal lung epithelial cells (BEAS-2B) and human lung adenocarcinoma cell lines A549 and H1299 was measured, respectively. See step 1 in this example for the specific detection method. The results were shown in FIG. 3. The results showed that CircCYB expression was higher in human lung adenocarcinoma cells compared with normal lung epithelial cells.

### Example 4: Construction of CircCYB-Knockdown Human Lung Adenocarcinoma Cell Lines A549 and H1299

The pLKO.1-puro lentiviral vector backbone plasmid was used to construct a plasmid expressing shRNA for knocking down CircCYB (named shCircCYB) with the target site sequence ACCAACTAACCCCCTAATA (SEQ ID NO: 2) and a plasmid expressing shRNA for knocking down Scramble (named shScramble, as a negative control) with the target site sequence CCTAAGGTTAAGTCGCCCTCG (SEQ ID NO: 3). The plasmids were combined with two lentiviral packaging vectors, pMD2g and psPAX2, and packaged in 293T cells. The specific steps are as follows:

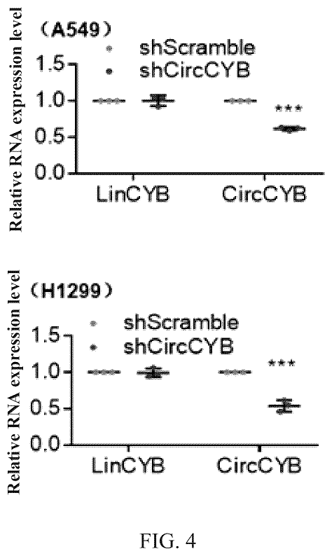

The engineered cell line 293T cells were used in the experiments to package the retrovirus. First, 293T cells were thawed and cultured in a 10 cm diameter dish with DMEM complete medium (DMEM high-glucose medium+10% NTC fetal bovine serum+100×MEM Non-Essential Amino Acids Solution+100×GlutMax supplement), and then passaged. After the 293T cells in the 100 mm cell culture dish reached 80%, the medium was replaced with fresh culture medium (DMEM complete medium, same below). In 1 mL of optimized medium (Opti-MEM, GIBICO, Cat. No.: 31985070), psPAX2:PMD2g:pLKO.1 were added in a ratio of 6 μg:2 μg:8 μg. After standing for 5 min, 4 times the volume of PEI transfection reagent was added, mixed well and allowed to stand for 12 min, and then the mixture was added to the above 293T cells. After 12 h of culture, the medium was replaced with fresh culture medium, and 36 hours later, the culture medium was collected, filtered through a 0.45 μm filter to remove 293T cells, and the first batch of viral infection liquid was supplemented to 10 mL with fresh DMEM complete medium. Fresh medium was added to the 293T cells after collection, and the second batch of 10 mL viral infection liquid was collected 12 hours later using the same method. A total of 24 mL of viral infection liquid was obtained and Polybrene (10 mg/mL, 10000x, to enhance viral infection) was added. Subsequently, 4 mL of the viral infection liquid was used to infect A549 and H1299 cells, and the medium was replaced with fresh DMEM complete medium daily. After 72 hours of culture, the A549 and H1299 cells were collected for RNA extraction and reverse transcription (the specific steps were the same as in Example 1), and the knockdown efficiency in human lung adenocarcinoma A549 and H1299 cells was validated using quantitative real-time PCR (the specific steps were the same as in Example 2). The results are shown in FIG. 4.

The results in FIG. 4 showed that shCircCYB effectively knocked down CircCYB in human lung adenocarcinoma A549 and H1299 cells compared to the negative control shScramble, without affecting the transcription of the linear parent gene MT-CYTB (i.e., LinCYTB) (for qPCR, forward primer: TTTCGCCCACTAAGCCAATC (SEQ ID NO: 11); reverse primer: GCCCATTTGAGTATTTTGTTT (SEQ ID NO: 12)), indicating the completed construction of the CircCYB-knockdown human lung adenocarcinoma A549 and H1299 cells.

### Example 5: Knockdown of CircCYB Significantly Promotes the Apoptosis in Human Lung Adenocarcinoma A549 and H1299 Cells

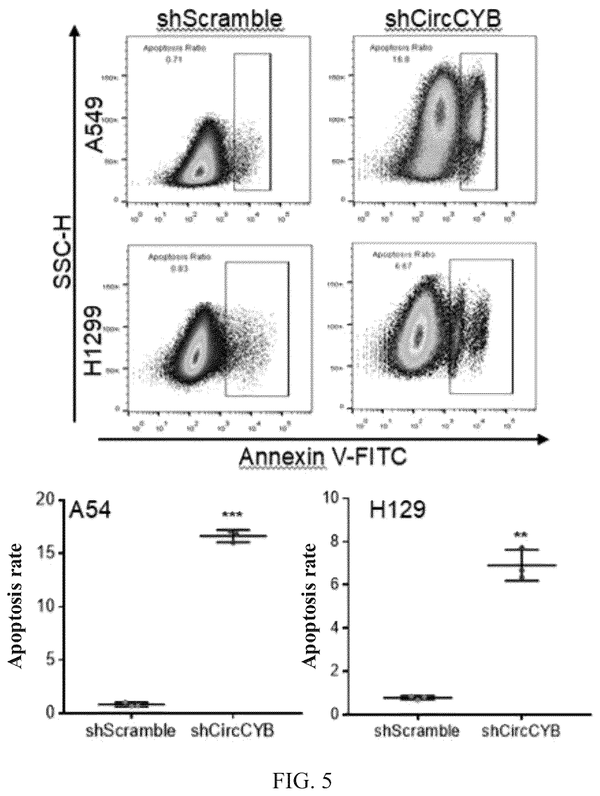

The apoptosis in human lung adenocarcinoma A549 and H1299 cells was detected on Day 4 after CircCYB knockdown using Biyuntian's Apoptosis Kit (Cat. No.: C1062L). Following the kit instructions, the adherent cells were washed with DPBS solution, and then digested with 0.25% trypsin (without EDTA) at room temperature. After 2 minutes, an equal volume of DMEM complete medium was added to terminate digestion, and the cells were transferred to a 15 mL centrifuge tube. The cells were centrifuged at 300×g for 3 minutes, and the supernatant was discarded. The cells were gently resuspended in DPBS and washed, followed by another centrifugation at 300×g for 3 minutes to obtain a cell pellet. The cell pellet was then resuspended in apoptosis staining working solution (containing 195 μL Annexin V-FITC binding solution and 5 μL Annexin V-FITC probe solution), gently mixed, and incubated at room temperature in the dark for 20 minutes. The cells were resuspended twice during incubation to improve staining efficiency. After staining, the tube was placed in an ice bath and immediately subjected to flow cytometric analysis. The results are shown in FIG. 5. The results indicated that, compared to the control cells (A549 and H1299 cells constructed with shScramble), knockdown of CircCYB significantly promoted apoptosis in human lung adenocarcinoma A549 and H1299 cells.

### Example 6: Knockdown of CircCYB does not Induce Apoptosis in Normal Lung Epithelial Cells

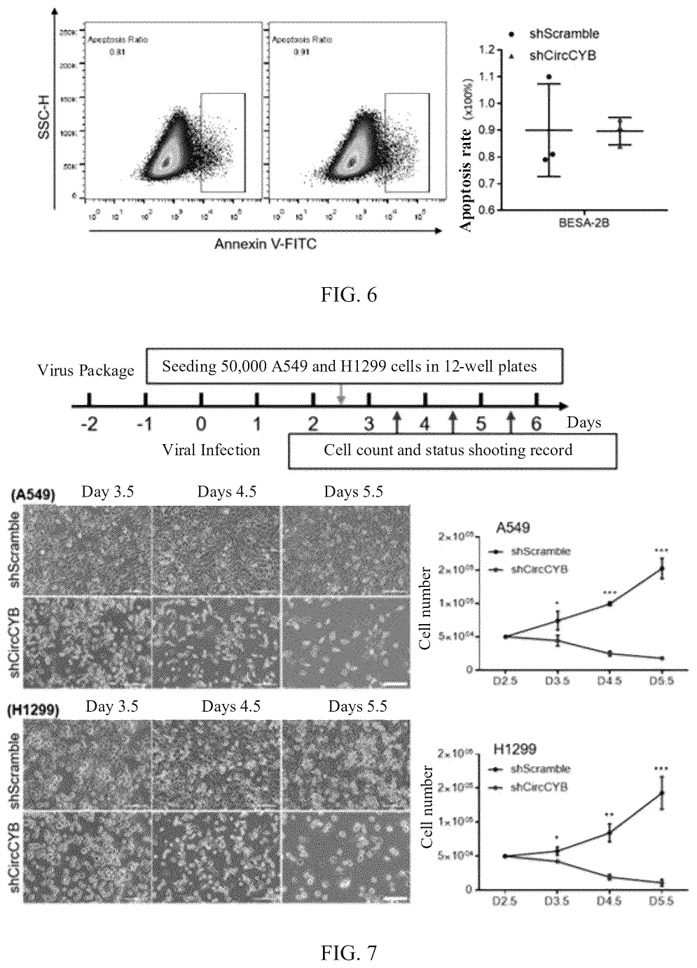

In order to further investigate whether knockdown of CircCYB affects the survival of normal lung epithelial cells, a normal lung epithelial cell line BEAS-2B with CircCYB knockdown was constructed (the specific steps were the same as in Example 4) for further apoptosis detection (the specific steps were the same as in Example 5). The results are shown in FIG. 6. The results showed that knockdown of CircCYB in normal lung epithelial cells did not induce apoptosis in normal lung epithelial cells, indicating that knockdown of CircCYB could induce apoptosis in lung adenocarcinoma cells without affecting the survival of normal lung epithelial cells. Therefore, it further demonstrates that the CircCYB of the present disclosure is expected to be a new target for the treatment of lung adenocarcinoma.

### Example 7: Knockdown of CircCYB Significantly Inhibits the Survival of Human Lung Adenocarcinoma A549 and H1299 Cells

To further explore the effect of CircCYB knockdown on the survival rate of human lung adenocarcinoma A549 and H1299 cells, the continuous survival of CircCYB-knockdown A549 and H1299 cells from Example 5 was monitored and recorded. First, cells infected with shScramble and shCircCYB viruses for 2.5 days were digested and counted. 50,000 cells were seeded in a 12-well cell culture plate, and the number and condition of the cells were recorded over 3 consecutive days. The results are shown in FIG. 7. The results demonstrated that, over time, knockdown of CircCYB significantly inhibited the survival of human lung adenocarcinoma A549 and H1299 cells, effectively killing the lung adenocarcinoma cells.

Although the embodiments of the present disclosure have been shown and described, it will be understood that the above-described embodiments are illustrative and not to be construed as limitations of the present disclosure. Changes, modifications, substitutions, and alterations may be made by those skilled in the art without departing from the scope of the present disclosure.

1. An isolated circular nucleic acid molecule having a nucleotide sequence as set forth in SEQ ID NO: 1.

2. An isolated protein or polypeptide encoded by the isolated circular nucleic acid molecule according to claim 1.

3. A construct carrying the isolated circular nucleic acid molecule according to claim 1.

4. The construct according to claim 3, wherein a vector of the construct is a non-pathogenic viral vector.

5. The construct according to claim 4, wherein the vector of the construct is an adenoviral vector, a lentiviral vector, or a retroviral vector.

6. A recombinant cell, carrying the isolated circular nucleic acid molecule according to claim 1.

7. A kit for detecting a cancer or tumor, the kit comprising:
a reagent used for detecting the circular nucleic acid molecule according to claim 1.

8. The kit according to claim 7, wherein the reagent comprises a probe, primer, or antibody for binding to the circular nucleic acid molecule.

9. The kit according to claim 7, wherein the cancer is selected from the group consisting of lung cancer, non-small cell lung cancer, papillary thyroid cancer, glioblastoma multiforme, colon cancer, rectal cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, pancreatic cancer, melanoma, head and neck squamous cell carcinoma, cervical cancer, skin cancer, glioma, esophageal cancer, oral squamous cell carcinoma, and gastric cancer.

10. A pharmaceutical composition, comprising a reagent inhibiting the expression or activity of the circular nucleic acid molecule according to claim 1.

11. The pharmaceutical composition according to claim 10, further comprising a pharmaceutically acceptable excipient.

12. The pharmaceutical composition according to claim 10, wherein the reagent comprises at least one of shRNA, siRNA, and CRISPR/Cas9 for knockdown of the circular nucleic acid molecule.

13. The pharmaceutical composition according to claim 12, wherein the reagent comprises a nucleotide sequence as set forth in SEQ ID NO: 2.

14. A method for promoting apoptosis in a cancer cell, comprising:
contacting a cancer cell expressing a circular nucleic acid molecule with a reagent for inhibiting the expression or activity of the circular nucleic acid molecule, wherein the circular nucleic acid molecule has a nucleotide sequence as set forth in SEQ ID NO: 1.

15. The method according to claim 14, wherein the reagent comprises at least one of shRNA, siRNA, and CRISPR/Cas9 for knockdown of the circular nucleic acid molecule.

16. The method according to claim 15, wherein the reagent comprises a nucleotide sequence as set forth in SEQ ID NO: 2.

17. A method for screening a medicament for treating or preventing cancer, the method comprising:
contacting a medicament to be screened with a cancer cell expressing a circular nucleic acid molecule having a nucleotide sequence as set forth in SEQ ID NO: 1, and
determining the medicament to be screened as a target medicament based on a decrease in the expression or activity of the circular nucleic acid molecule in the cancer cell after said contacting.

18. A method for preventing and/or treating a cancer or tumor, the method comprising:
administering to a subject a pharmaceutically acceptable amount of the pharmaceutical composition according to claim 10.

19. The method according to claim 18, wherein the cancer is selected from the group consisting of lung cancer, non-small cell lung cancer, papillary thyroid cancer, glioblastoma multiforme, colon cancer, rectal cancer, head and neck cancer, kidney cancer, bladder cancer, breast cancer, ovarian cancer, liver cancer, cholangiocarcinoma, sarcoma, acute myeloid leukemia, large cell neuroendocrine carcinoma, neuroblastoma, prostate cancer, pancreatic cancer, melanoma, head and neck squamous cell carcinoma, cervical cancer, skin cancer, glioma, esophageal cancer, oral squamous cell carcinoma, and gastric cancer.
